SEQUENCE LISTING (1 sequence)

SEQ ID NO: 1 - protein, 24 aa, Arabidopsis thaliana
FGSLVLNALPKGSRPGSGPSKKTN

(19) United States
(12) Patent Application Publication
(10) Pub. No.: US 2022/0400672 A1
(43) Pub. Date: Dec. 22, 2022

(54) IDL7 MATURE POLYPEPTIDE PLANT SENESCENCE ACCELERATOR, PREPARATION METHOD AND APPLICATION THEREOF

(71) Applicant: TOBACCO RESEARCH INSTITUTE OF CHINESE ACADEMY OF AGRICULTURAL SCIENCES, QINGDAO (CN)

(72) Inventors: YONGFENG GUO, QINGDAO (CN); CUN GUO, QINGDAO (CN); QI WANG, QINGDAO (CN); WEI LI, QINGDAO (CN); ZENGLIN ZHANG, QINGDAO (CN); LICHAO WEN, QINGDAO (CN); ZHICHAO DENG, QINGDAO (CN); TAO LIU, QINGDAO (CN)

(21) Appl. No.: 17/501,988

(22) Filed: Oct. 14, 2021

(30) Foreign Application Priority Data
Nov. 27, 2020 (CN) 202011363801.6

(51) Int. Cl. A01N 37/46; C07K 14/00

(52) U.S. Cl. CPC A01N 37/46; C07K 14/001

(57) ABSTRACT

The present application discloses a plant senescence accelerator named IDL7 mature polypeptide, and its preparation method and an application thereof. The application belongs to the field of a plant senescence accelerator, and the IDL7 mature polypeptide serves as a major functional ingredient with the following amino acid sequence: F-G-S-L-V-L-N-A-L-P-K-G-S-R-P-G-S-G-P-S-K-K-T-N. The IDL7 mature polypeptide plant senescence accelerator may promote the leaf senescence of plants without other additional adverse manifestations, thus this application has strong field operability.

### CROSS-REFERENCE TO RELATED APPLICATION

This application claims the priority benefits of Chinese Patent Application No. 202011363801.6 filed on Nov. 27, 2020 and entitled “IDL7 MATURE POLYPEPTIDE PLANT SENESCENCE ACCELERATOR, PREPARATION METHOD AND APPLICATION THEREOF”, the contents of the above identified application are hereby incorporated by reference in its entirety.

### TECHNICAL FIELD

The present application belongs to the field of a plant senescence accelerator, and particularly relates to an IDL7 mature polypeptide plant senescence accelerator and a preparation method and an application thereof.

### BACKGROUND OF THE INVENTION

Leaf senescence is an important factor influencing yield, quality and other agronomic characters of crops. Taking tobacco as an example, leaf is the main harvesting part of tobacco, leaves of the same plant undergo senescence at a sequential manner: leaves at lower positions of the stalk senesce earlier, followed by the middle and upper leaves. So far, the inconsistency of the senescence time of tobacco leaves is one of the main reasons that affect the harvest of leaves. While, the use of a senescence accelerator may effectively solve the problems of inconsistent maturity of tobacco leaves in different positions and regreening.

At present, the application of controlling plant senescence is mainly focused on the adjustment of plant hormones. For example, auxin and cytokinin may inhibit leaf senescence, while ethylene and abscisic acid may promote leaf senescence. Therefore, mastering the rule may artificially slow down the progress of leaf senescence. Senescence-associated functional parsing work has been carried out more widely and thoroughly, thus a lot of genes that promote leaf senescence are parsed functionally. Hu Youzhen, et al., have found that the deletion of 2-oxoglutarate dependent dioxygenase gene in Arabidopsis thaliana will cause delayed senescence phenotype; and using dexamethasone (DEX) to overexpress this gene can make Arabidopsis leaves prematurely senescence. Guo, et al., have found that the overexpression of an NAP gene, an Arabidopsis thaliana AtNAC family member may result in leaf senescence in advance, while the NAP gene deletion mutant may show a delayed senescence. In addition, the NAP gene has also been confirmed to affect the senescence of rice leaves. So far, it is rarely reported that senescence-associated genes have been directly applied in crop production.

Polypeptides are a kind of small molecules with regulatory effects in animals and plants; Mature polypeptides are post-translationally cleaved and modified from precursor proteins, and finally form functional molecules composed of shorter amino acids; As the ligands, the mature polypeptides recognize the receptor located on cytomembrane and initiates signal transduction. Mature polypeptides may be obtained by artificial synthesis, and have an influence on plant growth by directly external applying, such as the elongation of root. However, at present, there is a few of studies on the application of mature polypeptides in plants, especially crops, and even there is no study on the application of mature polypeptides in the promotion of plant senescence.

### SUMMARY OF THE INVENTION

The present application provides an IDL7 mature polypeptide plant senescence accelerator (hereafter refer as plant senescence accelerator), a preparation method and an application thereof. The IDL7 mature polypeptide plant senescence accelerator may promote the leaf senescence of plants without other additional adverse manifestations.

To solve the above technical problem, the present application provides an IDL7 mature polypeptide plant senescence accelerator, and an IDL7 mature polypeptide serves as a major functional ingredient in the plant senescence accelerator. Inflorescence definition in abscission (IDA)-like 7 is abbreviated as IDL7. The IDL7 mature polypeptide has the following amino acid sequence SEQ ID No. 1:

F-G-S-L-V-L-N-A-L-P-K-G-S-R-P-G-S-G-P-S-K-K-T-N

in which, A is the abbreviation of Ala; R is the abbreviation of Arg; N is the abbreviation of Asn; G is the abbreviation of Gly; L is the abbreviation of Leu; K is the abbreviation of Lys; F is the abbreviation of Phe, P is the abbreviation of Pro; S is the abbreviation of Ser; T is the abbreviation of Thr; V is the abbreviation of Val.

The material in the ASCI text file of the sequence listing of the above amino acid sequence SEQ ID No. 1 is herewith incorporated by reference. The ASCI text file was created on May 20, 2022 and has a file name “LPTF-108 Sequence Listing” and a size of 605 bytes.

In some embodiments, the IDL7 mature polypeptide plant senescence accelerator comprises the IDL7 mature polypeptide and a 2-(N-morpholine) ethanesulfonic acid solution; wherein, the 2-(N-morpholine) ethanesulfonic acid solution is prepared by dissolving 2-(N-morpholine) ethanesulfonic acid monohydrate into an MS fluid medium. It may be understood that, the 2-(N-morpholine) ethanesulfonic acid solution is used as the solvent of the IDL7 mature polypeptide since it is a new biological buffer that can better promote the polypeptide to provide corresponding functions.

In some embodiments, a concentration of the IDL7 mature polypeptide is 10-13 μmol/L, a concentration of a 2-(N-morpholine) ethanesulfonic acid solution is 2.8-3 mmol/L, and pH of the 2-(N-morpholine) ethanesulfonic acid solution is 5.8-5.9. It is understandable that the concentration of IDL7 mature polypeptide may be 10, 11, 12 and 13 μmol/L, the concentration of the 2-(N-morpholine) ethanesulfonic acid solution may be 2.8, 2.9 and 3 mmol/L, and pH of the 2-(N-morpholine) ethanesulfonic acid solution may be 5.8 and 5.9.

In some embodiments, the plant senescence accelerator also comprises assistants, the assistants may be a conventional assistants used in the art for leaf surface wetting and leaf surface extension. There is no specific limitation of the assistants in this technical solution, as long as the assistants can make solutions be extended and infiltrated on plant surface as soon as possible. It can be understood that the assistant may be Tween-20, and an addition amount of the assistant is 1-2 v/v‰. Tween-20 is a kind of surfactant, named as Polyoxyethylene dehydrated sorbitol monolaurate, CAS No. 9005-64-5.

The present application also provides a preparation method of an IDL7 mature polypeptide plant senescence accelerator, specifically, comprising following steps:

S1: dissolving IDL7 mature polypeptide powder into water to prepare an IDL7 mature polypeptide mother solution having a concentration of 10-13 mmol/L for subsequent use;

S2: adding quantitative 2-(N-morpholine) ethanesulfonic acid monohydrate into a MS fluid medium to prepare a 2-(N-morpholine) ethanesulfonic acid solution of 2.8-3 mmol/L, adjusting pH of the solution to 5.8-5.9, and after fully mixing and dissolving uniformly, the 2-(N-morpholine) ethanesulfonic acid solution is obtained;

MS medium powder (excluding agar and sucrose) used in the above steps is purchased from Beijing Solarbio company, when used, dissolve 4.42 g of the powder in 1 L water. The medium can fully dissolve 2-(N-morpholine) ethanesulfonic acid, ensuring that a certain osmotic potential is formed on the surface of a plant and laying the foundation for polypeptide to play its role; at the same time, the liquid medium can also be used as a solvent thus provide corresponding large and medium-sized element nutrition and guarantee the effect.

S3: adding the IDL7 mature polypeptide mother solution to the 2-(N-morpholine) ethanesulfonic acid solution, stirring uniformly and the IDL7 mature polypeptide plant senescence accelerator is obtained. In the IDL7 mature polypeptide plant senescence accelerator, a concentration of the IDL7 mature polypeptide is 10-13 μmol/L, a concentration of a 2-(N-morpholine) ethanesulfonic acid solution is 2.8-3 mmol/L.

It may be understood that, sequence of the above S1 and S2 may be replaced with each other, S1 may be performed first or S2 may be performed first, there are no specific requirements here. In addition, the prepared IDL7 mature polypeptide mother solution shall be used in time after preparation; if it is not used for more than 40 days, the mother solution shall be stored at −70° C.; if used within 40 days, the mother solution can be stored at −20° C. and stored at 4° C. within a week.

In some embodiments, after adding the obtained IDL7 mature polypeptide mother solution to 2-(N-morpholine) ethanesulfonic acid solution, the method further comprising a step of adding 1-2 v/v‰ Tween-20.

The present application also provides an application of the IDL7 mature polypeptide plant senescence accelerator of the above technical solutions in promoting plant senescence. When applying, the IDL7 mature polypeptide plant senescence accelerator is directly sprayed on leaf surface of plant leaves staying at full extension period, or using cotton balls soaked with the IDL7 mature polypeptide plant senescence accelerator to apply the leaf surface of plant leaves staying at full extension stage.

The present application also provides an application of the IDL7 mature polypeptide plant aging accelerator of the above technical solutions in promoting plant senescence. When applying, the IDL7 mature polypeptide plant aging accelerator is directly sprayed on leaf surface of tobacco leaves staying at early mature stage, or using cotton balls soaked with the IDL7 mature polypeptide plant senescence accelerator to apply the leaf surface of tobacco leaves staying at early mature stage.

It may be understood that no matter the plant senescence accelerator is directly sprayed on leaf surface of plant/crop leaves at early mature stage, or using cotton balls soaked with the plant senescence accelerator to apply the leaf surface of plant/crop leaves at early mature stage, the above applying process is completed on condition that the plant growth situation is not influenced, and meanwhile, may promote the plant leaf senescence; particularly, the applying of the plant senescence accelerator is beneficial to the consistent harvest of the crop with desynchronized senescence leaves, for example, tobacco. It may be understood that a concentration of the plant senescence accelerator directly sprayed on the leaf surface of plant/crop leaves at early mature stage is 10-12 μmol/L.

Positive advantages and beneficial effects achieved by the present application are as follows:

1. IDL7 mature polypeptide in the IDL7 mature polypeptide plant senescence accelerator provided by the present application is synthesized artificially, it is easy to get and may be synthesized in batches.

2. The method for preparing and using the plant senescence promoter provided by the present application is simple and has strong field operability. It does not require special training for the application personnel, and the operation is convenient and practical;

3. The plant senescence accelerator provided by the present application has moderate influences on leaf senescence (compared with normal senescent leaves, there is no significant difference). And it will cause no adverse reaction, but bring beneficial effects.

### BRIEF DESCRIPTION OF THE DRAWINGS

FIGS. 1A-1B are schematic diagrams showing a result of Arabidopsis thaliana detached leaves treated by an artificially synthesized IDL7 mature polypeptide plant senescence accelerator;

FIGS. 2A-2B are schematic diagrams showing a result of in vivo tobacco leaves treated by an IDL7 mature polypeptide plant senescence accelerator.

### DETAILED DESCRIPTION OF THE INVENTION

To more clearly and specifically introduce the IDL7 mature polypeptide plant senescence accelerator and its preparation method and application provided by the embodiments of the present application the technical solution in the embodiments of the present application will be further described clearly and integrally. Apparently, the examples described herein are merely a portion of embodiments of the present application, rather than all the embodiments. Based on the embodiments of the present application, all the other embodiments obtained by a person skilled in the art without inventive effort shall fall within the protection scope of the present application.

It is to be noted that the term “mother solution” used in the embodiments refers to a solution with a higher concentration, and the solution needs to be diluted in subsequent use to serve as a working solution with a lower concentration.

### Embodiment 1

An IDL7 mature polypeptide plant senescence accelerator comprising 10 μmol/L IDL7 mature polypeptide, a 2.8 mmol/L 2-(N-morpholine) ethanesulfonic acid solution; the 2-(N-morpholine) ethanesulfonic acid solution was prepared by dissolving 2-(N-morpholine) ethanesulfonic acid monohydrate into an MS fluid medium, and the 2-(N-morpholine) ethanesulfonic acid solution has a pH value of 5.8.

The preparation method is as follows:

(1) IDL7 mature polypeptide powder was weighed and dissolved into water, and an IDL7 mature polypeptide mother solution having a concentration of 10 mmol/L was prepared for further use;

(2) 2-(N-morpholine) ethanesulfonic acid monohydrate was weighed quantitatively and added to the MS fluid medium, and the 2.8 mmol/L 2-(N-morpholine) ethanesulfonic acid solution is prepared, pH of the solution was adjusted to 5.8, and materials were fully mixed and dissolved uniformly to obtain the 2-(N-morpholine) ethanesulfonic acid solution;

(3) 40 μL of the IDL7 mature polypeptide mother solution obtained in the step (1) were taken and added to 40 mL of the 2-(N-morpholine) ethanesulfonic acid solution prepared in the step (2), and stirred uniformly with a glass rod to obtain 40 mL of the IDL7 mature polypeptide plant senescence accelerator.

### Embodiment 2

An IDL7 mature polypeptide plant senescence accelerator comprising 12 μmol/L IDL7 mature polypeptide, a 2.9 mmol/L 2-(N-morpholine) ethanesulfonic acid solution; the 2-(N-morpholine) ethanesulfonic acid solution was prepared by dissolving 2-(N-morpholine) ethanesulfonic acid monohydrate into an MS fluid medium, and the 2-(N-morpholine) ethanesulfonic acid solution has a pH value of 5.9.

The preparation method was as follows:

(1) IDL7 mature polypeptide powder was weighed and dissolved into water, and an IDL7 mature polypeptide mother solution having a concentration of 10 mmol/L was prepared for further use;

(2) 2-(N-morpholine) ethanesulfonic acid monohydrate was weighed quantitatively and added to the MS fluid medium, and the 2.9 mmol/L 2-(N-morpholine) ethanesulfonic acid solution is prepared, pH of the solution was adjusted to 5.9, and materials were fully mixed and dissolved uniformly to obtain the 2-(N-morpholine) ethanesulfonic acid solution;

(3) 120 μL of the IDL7 mature polypeptide mother solution obtained in the step (1) were taken and added to 100 mL of the 2-(N-morpholine) ethanesulfonic acid solution prepared in the step (2), and stirred uniformly with a glass rod to obtain 100 mL of the IDL7 mature polypeptide plant senescence accelerator.

### Embodiment 3

An IDL7 mature polypeptide plant senescence accelerator comprising 13 μmol/L IDL7 mature polypeptide, a 3 mmol/L 2-(N-morpholine) ethanesulfonic acid solution; the 2-(N-morpholine) ethanesulfonic acid solution was prepared by dissolving 2-(N-morpholine) ethanesulfonic acid monohydrate into an MS fluid medium, and the 2-(N-morpholine) ethanesulfonic acid solution has a pH value of 5.8.

The preparation method was as follows:

(1) IDL7 mature polypeptide powder was weighed and dissolved into water, and an IDL7 mature polypeptide mother solution having a concentration of 10 mmol/L was prepared for further use;

(2) 2-(N-morpholine) ethanesulfonic acid monohydrate was weighed quantitatively and added to the MS fluid medium, and the 3 mmol/L 2-(N-morpholine) ethanesulfonic acid solution is prepared, pH of the solution was adjusted to 5.8, and materials were fully mixed and dissolved uniformly to obtain the 2-(N-morpholine) ethanesulfonic acid solution;

(3) 78 μL of the IDL7 mature polypeptide mother solution obtained in the step (1) were taken and added to 60 mL of the 2-(N-morpholine) ethanesulfonic acid solution prepared in the step (2), and stirred uniformly with a glass rod to obtain 60 mL of the IDL7 mature polypeptide plant senescence accelerator.

Performance Test

Laboratory Test

Embodiment 1 was set as an example; the IDL7 mature polypeptide plant senescence accelerator prepared in Embodiment 1 was used to treat detached leaves of Arabidopsis thaliana and in vivo leaves of tobacco respectively according to method of applying.

Treatment of Arabidopsis thaliana leaves: Arabidopsis thaliana grew for 30 days around under continuous illumination to obtain detached leaves from the same leaf position (the 6th leaf position); the detached leaves were treated by a puncher having a diameter of 0.5 cm, then placed flat on a petri dish; treated with 2.8 mmol/L 2-(N-morpholine) ethanesulfonic acid solution (hereafter refer as MES) as a control group; meanwhile, 10 μmol/L plant senescence accelerator was evenly and directly sprayed on the detached leaf surface of Arabidopsis thaliana as an experimental group, and placed the leaves of the control group and the experimental group for 4 days, then phenotype was observed.

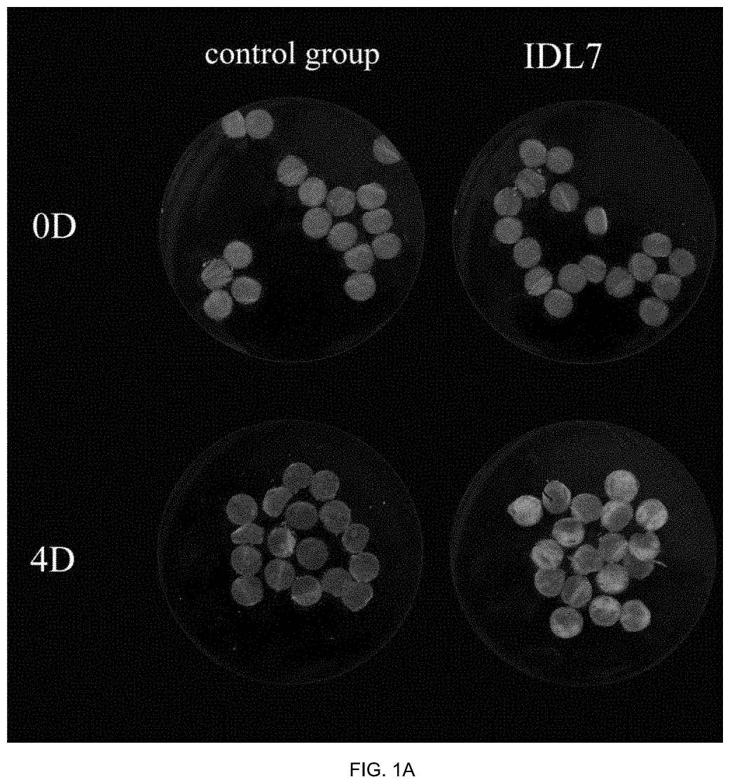

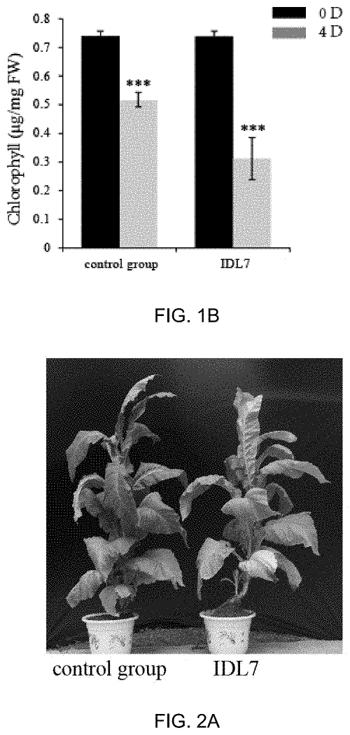

As shown in FIG. 1A, Arabidopsis thaliana leaves with the same growth vigor in the same leaf position (the 6th leaf position) were selected, and treated by a puncher with a diameter of 0.5 cm. Then the Arabidopsis thaliana leaves were put on a Petri dish regularly, and treated by spraying with the IDL7 mature polypeptide plant senescence accelerator to obtain the experimental group, meanwhile, the control group was added and treated by the same method except that the plant senescence accelerator was replaced by 2.8 mmol/L EMS. The leaves of the control group and the experimental group were put for 4 days under continuous illumination.

It can be seen from the results that the IDL7 mature polypeptide plant senescence accelerator may promote the leaf senescence of Arabidopsis thaliana. 4 days later, the Arabidopsis thaliana detached leaves sprayed with the IDL7 mature polypeptide plant senescence accelerator had obvious premature phenotype. Meanwhile, chlorophyll content was measured by ethanol method, as shown in FIG. 1B; compared with the control group, the chlorophyll content in the leaves treated by the IDL7 mature polypeptide plant senescence accelerator decreased significantly, and the chlorophyll content in the control group decreased to 0.517 μg/mg, and the chlorophyll content in the treatment group decreased to 0.311 μg/mg.

Field Test

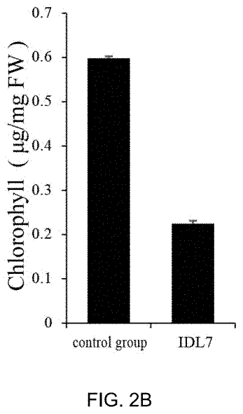

As shown in FIG. 2A, the tobacco variety K326 cultivated for about 60 days in field conditions served as a material, and was sprayed with 10 μmol/L IDL7 mature polypeptide plant senescence accelerator. Meanwhile, a control group was sprayed with a solution free of IDL7 mature polypeptide; 14 days later, it can be obviously observed that the tobacco sprayed with the IDL7 mature polypeptide plant senescence accelerator showed premature phenotype. Meanwhile, ethanol method was used to measure the chlorophyll content of tobacco leaves being sprayed with the IDL7 mature polypeptide plant senescence accelerator and the tobacco leaves of the control group after 14 days; and the results showed that compared with the control group (the chlorophyll content of the control group is 0.598 μg/mg), as shown in FIG. 2B, the chlorophyll content of the tobacco leaves treated by the IDL7 mature polypeptide plant senescence accelerator is 0.225 μg/mg, which is decreased significantly. The results showed that the IDL7 mature polypeptide plant senescence accelerator had obvious promotion effect on the leaf senescence of in vivo plants indeed.

1. An IDL7 mature polypeptide plant senescence accelerator, wherein an IDL7 mature polypeptide serves as a major functional ingredient, and the IDL7 mature polypeptide has the following amino acid sequence SEQ ID No. 1:
F-G-S-L-V-L-N-A-L-P-K-G-S-R-P-G-S-G-P-S-K-K-T-N;
the IDL7 mature polypeptide plant senescence accelerator comprises the IDL7 mature polypeptide and a 2-(N-morpholine) ethanesulfonic acid solution; wherein, the 2-(N-morpholine) ethanesulfonic acid solution is prepared by dissolving 2-(N-morpholine) ethanesulfonic acid monohydrate into an MS fluid medium;
a concentration of the IDL7 mature polypeptide is 10-13 μmol/L, a concentration of the 2-(N-morpholine) ethanesulfonic acid solution is 2.8-3 mmol/L, and pH value of the 2-(N-morpholine) ethanesulfonic acid solution is 5.8-5.9.

2. The IDL7 mature polypeptide plant senescence accelerator according to claim 1, wherein a concentration of the IDL7 mature polypeptide is 10 μmol/L, a concentration of the 2-(N-morpholine) ethanesulfonic acid solution is 2.8 mmol/L, and the pH value of the 2-(N-morpholine) ethanesulfonic acid solution is 5.8.

3. The IDL7 mature polypeptide plant senescence accelerator according to claim 1, further comprising an assistant, the assistant comprises Tween-20, and an addition amount of the assistant is 1-2 v/v‰.

4. A preparation method of the IDL7 mature polypeptide plant senescence accelerator of claim 1, comprising following steps:
dissolving IDL7 mature polypeptide powder into water to prepare an mother solution of IDL7 mature polypeptide with a concentration of 10 mmol/L for subsequent use;
adding 2-(N-morpholine) ethanesulfonic acid monohydrate into a MS fluid medium to prepare a 2-(N-morpholine) ethanesulfonic acid solution of 2.8-3 mmol/L, adjusting pH of the solution to 5.8-5.9, and after fully mixing and dissolving, the 2-(N-morpholine) ethanesulfonic acid solution is obtained;
adding the IDL7 mature polypeptide mother solution to the 2-(N-morpholine) ethanesulfonic acid solution, stirring uniformly and the IDL7 mature polypeptide plant senescence accelerator is obtained.

5. The preparation method of claim 4, wherein after adding the obtained IDL7 mature polypeptide mother solution to the 2-(N-morpholine) ethanesulfonic acid solution, the method further comprises a step of adding 1-2 v/v‰ Tween-20.

6. An application of the IDL7 mature polypeptide plant senescence accelerator according to claim 1 in promoting plant senescence, comprising the following steps:
directly spraying the IDL7 mature polypeptide plant senescence accelerator on leaf surface of plant leaves staying at full extension period, or using cotton balls soaked with the IDL7 mature polypeptide plant senescence accelerator to apply the leaf surface of plant leaves staying at full extension stage.

7. An application of the IDL7 mature polypeptide plant senescence accelerator according to claim 2 in promoting plant senescence, comprising the following steps:
directly spraying the IDL7 mature polypeptide plant senescence accelerator on leaf surface of plant leaves staying at full extension period, or using cotton balls soaked with the IDL7 mature polypeptide plant senescence accelerator to apply the leaf surface of plant leaves staying at full extension stage.

8. An application of the IDL7 mature polypeptide plant senescence accelerator according to claim 3 in promoting plant senescence, comprising the following steps:
directly spraying the IDL7 mature polypeptide plant senescence accelerator on leaf surface of plant leaves staying at full extension period, or using cotton balls soaked with the IDL7 mature polypeptide plant senescence accelerator to apply the leaf surface of plant leaves staying at full extension stage.

9. An application of the IDL7 mature polypeptide plant senescence accelerator according to claim 1 in promoting plant senescence, comprising the following steps:
directly spraying the IDL7 mature polypeptide plant aging accelerator is directly sprayed on leaf surface of tobacco leaves staying at early mature stage, or using cotton balls soaked with the IDL7 mature polypeptide plant senescence accelerator to apply the leaf surface of tobacco leaves staying at early mature stage.

10. An application of the IDL7 mature polypeptide plant senescence accelerator according to claim 2 in promoting plant senescence, comprising the following steps:
directly spraying the IDL7 mature polypeptide plant aging accelerator is directly sprayed on leaf surface of tobacco leaves staying at early mature stage, or using cotton balls soaked with the IDL7 mature polypeptide plant senescence accelerator to apply the leaf surface of tobacco leaves staying at early mature stage.

11. An application of the IDL7 mature polypeptide plant senescence accelerator according to claim 3 in promoting plant senescence, comprising the following steps:
directly spraying the IDL7 mature polypeptide plant aging accelerator is directly sprayed on leaf surface of tobacco leaves staying at early mature stage, or using cotton balls soaked with the IDL7 mature polypeptide plant senescence accelerator to apply the leaf surface of tobacco leaves staying at early mature stage.

12. The application according to claim 6, wherein a concentration of the plant senescence accelerator directly sprayed on the leaf surface is 10-12 μmol/L.

13. The application according to claim 9, wherein a concentration of the plant senescence accelerator directly sprayed on the leaf surface is 10-12 μmol/L.
